(19) United States
(12) Patent Application Publication
(10) Pub. No.: US 2026/0311458 A1
(43) Pub. Date: Oct. 8, 2026

(54) HARDWARE APPARATUS FOR SELECTING RADIOMIC FEATURES, METHOD FOR BUILDING A LEARNING MODEL USING SIGNIFICANT RADIOMIC FEATURES, AND METHOD FOR SUPPORTING PATIENT TREATMENT USING A LEARNING MODEL

(71) Applicant: SAMSUNG LIFE PUBLIC WELFARE FOUNDATION, Seoul (KR)

(72) Inventors: Woo Kyoung JEONG, Seoul (KR); Kyung A KIM, Seoul (KR); Sung Soo HONG, Seoul (KR); Sung Jun HONG, Suwon-si (KR)

(21) Appl. No.: 19/640,180

(22) Filed: Apr. 6, 2026

Related U.S. Application Data
(63) Continuation-in-part of application No. PCT/KR2023/020062, filed on Dec. 7, 2023.

(30) Foreign Application Priority Data
Oct. 6, 2023 (KR) 10-2023-0133813

(51) Int. Cl. G06V 10/771; G06T 7/00; G06V 10/77; G06V 10/774; G16H 20/00

(52) U.S. Cl. CPC G06V 10/771; G06T 7/0012; G06V 10/7715; G06V 10/774; G16H 20/00; G06T 2207/10081; G06T 2207/10088; G06T 2207/10104; G06T 2207/10116; G06T 2207/10132; G06T 2207/20081; G06T 2207/30004; G06V 2201/03

(57) ABSTRACT

A method for selecting radiomic features for building an artificial intelligence model includes performing, by a hardware apparatus, univariate analysis on each of a plurality of radiomic features extracted from medical images with respect to an output value of a learning model to compute a statistical relevance indicator, removing, by the hardware apparatus, features whose relevance to the output value is below a threshold from among the radiomic features based on the statistical relevance indicator, removing, by the hardware apparatus, features having collinearity above a threshold between features from among the radiomic features, additionally removing by the hardware apparatus, features whose relevance to the output value is below a threshold from among the radiomic features based on the statistical relevance indicator, and applying, by the hardware apparatus, an embedded method to the remaining radiomic features to select final radiomic features.

### CROSS REFERENCE TO RELATED APPLICATIONS

This application is a Continuation of PCT International Application No. PCT/KR2023/020062, filed on Dec. 7, 2023, which claims priority to Korean Patent Application No. 10-2023-0133813, filed on Oct. 6, 2023, the entire disclosures of which are incorporated herein by reference for all purposes.

### BACKGROUND

### 1. Field

The present disclosure relates to a technique for selecting significant radiomic features from medical images. The present disclosure also relates to a technique for building a learning model using significant radiomic features. The present disclosure further relates to a technique for supporting patient treatment using a built learning model.

### 2. Description of the Related Art

In the medical field, images are representative data for diagnosing diseases and predicting prognoses. Representative medical images include ultrasound images, X-rays, CT (Computed Tomography) scans, PET (Positron Emission Tomography) scans using radioactive isotopes, and MRI ((Magnetic Resonance Imaging) scans using magnetic resonance phenomena.

Radiomics analysis refers to research that extracts quantitative and statistical features related to diseases from image data such as ultrasound images, CT scans, PET scans, and MRI scans, and performs disease diagnosis and classification based on the extracted features. Radiomic features are composed of various factors across multiple categories, such as shape, intensity, texture, high-order, and filter. Hundreds to thousands of radiomic features may be extracted from a single medical image.

Recently, applications utilizing AI (artificial intelligence) such as machine learning have emerged in the medical field. An AI model may receive radiomic features extracted from medical images as input and classify lesions. As described above, radiomic features are composed of a large number of items. Therefore, prior to building an AI model, significant radiomic features for a specific AI model must be pre-selected in order to prevent overfitting, improve model performance, and reduce model-building time and cost.

The description of the related art should not be assumed to be prior art merely because it is mentioned in or associated with this section. The description of the related art includes information that describes one or more aspects of the subject technology, and the description in this section does not limit the invention.

### SUMMARY

In one or more aspects of the present disclosure, a method for supporting patient treatment using a learning model includes receiving, by an electronic device, a medical image of a patient, extracting, by the electronic device, radiomic features from the medical image, inputting, by the electronic device, the extracted radiomic features into a pre-trained learning model to generate clinical information about the patient, providing, by the electronic device, the clinical information to a user terminal, and selecting a treatment modality for the patient or establishing a treatment plan based on the clinical information. The learning model is trained using significant radiomic features. The process of selecting the significant radiomic features includes: (i) a first filtering based on a filter method applied to radiomic features extracted from medical images as training data; (ii) a second filtering that removes redundant features based on collinearity from the candidate radiomic features remaining after the first filtering; (iii) a third filtering based on a filter method additionally incorporating an odds ratio criterion applied to the candidate radiomic features remaining after the second filtering; and (iv) selecting based on an embedded method applied to the candidate radiomic features remaining after the third filtering.

In one or more aspects of the present disclosure, a method for building a learning model using radiomic features includes receiving, by a hardware apparatus, a plurality of radiomic features extracted from medical images, generating, by the hardware apparatus, a feature map including at least one statistical relevance indicator and at least one collinearity indicator for each of the radiomic features, selecting, by the hardware apparatus, significant radiomic features by removing some of the radiomic features using the feature map, and training, by the hardware apparatus, a learning model using the significant radiomic features.

In one or more aspects of the present disclosure, a hardware apparatus includes a storage device configured to store medical images, a memory configured to store instructions for radiomic feature filtering, and a processor configured to execute the instructions to perform the following operations: (i) extracting a plurality of radiomic features from the medical images; (ii) performing univariate statistical analysis on each of the radiomic features with respect to an output value of a learning model to compute one or more statistical relevance indicators; (iii) performing a first removal of features whose relevance to the output value is below a threshold, from a candidate radiomic feature set including the radiomic features, based on at least one of the statistical relevance indicators; (iv) performing a second removal of redundant features from the candidate radiomic feature set remaining after the first removal based on collinearity criteria; (v) performing a third removal of features whose relevance to the output value is below a threshold, from the candidate radiomic feature set remaining after the second removal, based on at least one of the statistical relevance indicators; and (vi) applying an embedded method to the candidate radiomic feature set remaining after the third removal to select final radiomic features.

Additional features, advantages, and aspects of the present disclosure are set forth in part in the description that follows and in part will become apparent from the present disclosure or may be learned by practice of the inventive concepts provided herein. Other features, advantages, and aspects of the present disclosure may be realized and attained by the descriptions provided in the present disclosure, or derivable therefrom, and the claims hereof as well as the drawings. It is intended that all such features, advantages, and aspects be included within this description, be within the scope of the present disclosure, and be protected by the following claims. Nothing in this section should be taken as a limitation on those claims. Further aspects and advantages are discussed below in conjunction with embodiments of the present disclosure.

It is to be understood that both the foregoing description and the following description of the present disclosure are examples, and are intended to provide further explanation of the disclosure as claimed.

### BRIEF DESCRIPTION OF THE DRAWINGS

The accompanying drawings, which are included to provide a further understanding of the present disclosure, are incorporated in and constitute a part of this present disclosure, illustrate aspects and embodiments of the present disclosure, and together with the description serve to explain principles and examples of the disclosure. In the drawings:

FIG. 1 is an example of a process for building a learning model using radiomic features.

FIG. 2 is an example of a process for selecting radiomic features.

FIG. 3 is an example of a combined process for selecting radiomic features.

FIG. 4 illustrates a radiomic feature selection process using an actual dataset.

FIG. 5 is an example of a hardware apparatus for selecting radiomic features.

FIG. 6 is an example of a subject diagnosis system using a learning model that takes radiomic features as input data.

Throughout the drawings and the detailed description, unless otherwise described, the same drawing reference numerals should be understood to refer to the same elements, features, and structures. The sizes of regions and elements, and depiction thereof, may be exaggerated for clarity, illustration, and/or convenience.

### DETAILED DESCRIPTION

The following detailed description is provided to assist the reader in gaining a comprehensive understanding of the methods, apparatuses, and/or systems described herein. Accordingly, various changes, modifications, and equivalents of the systems, apparatuses and/or methods described herein will be understood by those of ordinary skill in the art.

Moreover, descriptions of well-known functions and constructions may be omitted for increased clarity and conciseness. Further, repetitive descriptions may be omitted for brevity. The progression of processing steps and/or operations described is a non-limiting example.

The sequence of steps and/or operations is not limited to that set forth herein and may be changed to occur in an order that is different from an order described herein, with the exception of steps and/or operations necessarily occurring in a particular order. In one or more examples, two operations in succession may be performed substantially concurrently, or the two operations may be performed in a reverse order or in a different order depending on a function or operation involved.

Unless stated otherwise, like reference numerals may refer to like elements throughout even when they are shown in different drawings. Unless stated otherwise, the same reference numerals may be used to refer to the same or substantially the same elements throughout the specification and the drawings. In one or more aspects, identical elements (or elements with identical names) in different drawings may have the same or substantially the same functions and properties unless stated otherwise. Names of the respective elements used in the following explanations are selected only for convenience and may be thus different from those used in actual products.

Advantages and features of the present disclosure, and implementation methods thereof, are clarified through the embodiments described with reference to the accompanying drawings. The present disclosure may, however, be embodied in different forms and should not be construed as limited to the embodiments set forth herein. Rather, these embodiments are examples and are provided so that this disclosure may be thorough and complete to assist those skilled in the art to understand the inventive concepts without limiting the protected scope of the present disclosure.

Shapes, dimensions (e.g., sizes, lengths, locations, and areas), proportions, ratios, numbers, the number of elements, and the like disclosed herein, including those illustrated in the drawings, are merely examples, and thus, the present disclosure is not limited to the illustrated details. It is, however, noted that the relative dimensions of the components illustrated in the drawings are part of the present disclosure.

When the term “comprise,” “have,” “include,” “contain,” “constitute,” “made of,” “formed of” “composed of,” or the like is used with respect to one or more elements (e.g., components, structures, groups, circuits, networks, members, parts, areas, portions, integers, steps, operations, and/or the like), one or more other elements may be added unless a term such as “only” or the like is used. The terms used in the present disclosure are merely used in order to describe particular example embodiments, and are not intended to limit the scope of the present disclosure. The terms of a singular form may include plural forms unless the context clearly indicates otherwise. For example, an element may be one or more elements. An element may include a plurality of elements. The word “exemplary” is used to mean serving as an example or illustration. Embodiments are example embodiments. Aspects are example aspects. In one or more implementations, “embodiments,” “examples,” “aspects,” and the like should not be construed to be preferred or advantageous over other implementations. An embodiment, an example, an example embodiment, an aspect, or the like may refer to one or more embodiments, one or more examples, one or more example embodiments, one or more aspects, or the like, unless stated otherwise. Further, the term “may” encompasses all the meanings of the term “can.”

In one or more aspects, unless explicitly stated otherwise, an element, feature, or corresponding information (e.g., a level, range, dimension, or the like) is construed to include an error or tolerance range even where no explicit description of such an error or tolerance range is provided. An error or tolerance range may be caused by various factors (e.g., process factors, internal or external impact, noise, or the like). In interpreting a numerical value, the value is interpreted as including an error range unless explicitly stated otherwise.

When a positional relationship between two elements (e.g., components, structures, groups, circuits, networks, members, parts, areas, portions, and/or the like) are described using any of the terms such as “adjacent to,” “beside,” “next to,” and/or the like indicating a position or location, one or more other elements may be located between the two elements unless a more limiting term, such as “immediate(ly),” “direct(ly),” or “close(ly),” is used. Furthermore, the spatially relative terms such as the foregoing terms as well as other terms such as “column,” “row,” “vertical,” “horizontal,” “diagonal,” and the like refer to an arbitrary frame of reference.

In describing a temporal relationship, when the temporal order is described as, for example, “after,” “following,” “subsequent,” “next,” “before,” “preceding,” “prior to,” or the like, a case that is not consecutive or not sequential may be included and thus one or more other events may occur therebetween, unless a more limiting term, such as “just,” “immediate(ly),” or “direct(ly),” is used.

It is understood that, although the terms “first,” “second,” and the like may be used herein to describe various elements (e.g., components, structures, groups, circuits, networks, members, parts, areas, portions, and/or the like), these elements should not be limited by these terms, for example, to any particular order, precedence, or number of elements. These terms are used only to distinguish one element from another. For example, a first element may denote a second element, and, similarly, a second element may denote a first element, without departing from the scope of the present disclosure. Furthermore, the first element, the second element, and the like may be arbitrarily named according to the convenience of those skilled in the art without departing from the scope of the present disclosure. For clarity, the functions or structures of these elements (e.g., the first element, the second element, and the like) are not limited by ordinal numbers or the names in front of the elements. Further, a first element may include one or more first elements. Similarly, a second element or the like may include one or more second elements or the like.

In describing elements of the present disclosure, the terms “first,” “second,” “A,” “B,” “(a),” “(b),” or the like may be used. These terms are intended to identify the corresponding element(s) from the other element(s), and these are not used to define the essence, basis, order, or number of the elements.

The expression that an element (e.g., component, structure, group, circuit, network, member, part, area, portion, and/or the like) “is engaged” with another element may be understood, for example, as that the element may be either directly or indirectly engaged with the another element. The term “is engaged” or similar expressions may refer to a term such as “is connected,” “is coupled,” “is combined,” “is linked,” “is provided,” “interacts,” or the like. The engagement may involve one or more intervening elements disposed or interposed between the element and the another element, unless otherwise specified.

The terms such as a “line” or “direction” should not be interpreted only based on a geometrical relationship in which the respective lines or directions are parallel, perpendicular, diagonal, or slanted with respect to each other, and may be meant as lines or directions having wider directivities within the range within which the components of the present disclosure may operate functionally.

The term “at least one” should be understood as including any and all combinations of one or more of the associated listed items. For example, each of the phrases “at least one of a first item, a second item, or a third item” and “at least one of a first item, a second item, and a third item” may represent (i) a combination of items provided by two or more of the first item, the second item, and the third item or (ii) only one of the first item, the second item, or the third item. Further, at least one of a plurality of elements can represent (i) one element of the plurality of elements, (ii) some elements of the plurality of elements, or (iii) all elements of the plurality of elements. Further, “at least some,” “at least some portions,” “at least some parts,” “at least a portion,” “at least one or more portions,” “at least a part,” “at least one or more parts,” “at least some elements,” “one or more,” or the like of a plurality of elements can represent (i) one element of the plurality of elements, (ii) a portion (or a part) of the plurality of elements, (iii) one or more portions (or parts) of the plurality of elements, (iv) multiple elements of the plurality of elements, or (v) all of the plurality of elements. Moreover, “at least some,” “at least some portions,” “at least some parts,” “at least a portion,” “at least one or more portions,” “at least a part,” “at least one or more parts,” or the like of an element can represent (i) a portion (or a part) of the element, (ii) one or more portions (or parts) of the element, or (iii) the element, or all portions of the element.

The expression of a first element, a second elements “and/of” a third element should be understood as one of the first, second and third elements or as any or all combinations of the first, second and third elements. By way of example, A, B and/or C may refer to only A; only B; only C; any of A, B, and C (e.g., A, B, or C); some combination of A, B, and C (e.g., A and B; A and C; or B and C); or all of A, B, and C. Furthermore, an expression “A/B” may be understood as A and/or B. For example, an expression “A/B” may refer to only A; only B; A or B; or A and B.

In one or more aspects, the terms “between” and “among” may be used interchangeably simply for convenience unless stated otherwise. For example, an expression “between a plurality of elements” may be understood as among a plurality of elements. In another example, an expression “among a plurality of elements” may be understood as between a plurality of elements. In one or more examples, the number of elements may be two. In one or more examples, the number of elements may be more than two. Furthermore, when an element is referred to as being“between” at least two elements, the element may be the only element between the at least two elements, or one or more intervening elements may also be present.

In one or more aspects, the phrases “each other” and “one another” may be used interchangeably simply for convenience unless stated otherwise. For example, an expression “different from each other” may be understood as being different from one another. In another example, an expression “different from one another” may be understood as being different from each other. In one or more examples, the number of elements involved in the foregoing expression may be two. In one or more examples, the number of elements involved in the foregoing expression may be more than two.

In one or more aspects, the phrases “one or more among” and “one or more of” may be used interchangeably simply for convenience unless stated otherwise.

The term “or” means “inclusive or” rather than “exclusive or.” That is, unless otherwise stated or clear from the context, the expression that “x uses a or b” means any one of natural inclusive permutations. For example, “a or b” may mean “a,” “b,” or “a and b.” For example, “a, b or c” may mean “a,” “b,” “c,” “a and b,” “b and c,” “a and c,” or “a, b and c.”

A phrase “substantially the same” may indicate a degree of being considered as being equivalent to each other taking into account minute differences due to errors in the manufacturing or operating process.

Features of various embodiments of the present disclosure may be partially or entirely coupled to or combined with each other, may be technically associated with each other, and may be variously operated, linked or driven together in various ways. Embodiments of the present disclosure may be implemented or carried out independently of each other or may be implemented or carried out together in a co-dependent or related relationship. In one or more aspects, the components of each apparatus and device according to various embodiments of the present disclosure are operatively coupled and configured.

The terms used herein have been selected as being general in the related technical field; however, there may be other terms depending on the development and/or change of technology, convention, preference of technicians, and so on. Therefore, the terms used herein should not be understood as limiting technical ideas, but should be understood as examples of the terms for describing example embodiments.

Further, in a specific case, a term may be arbitrarily selected by an applicant, and in this case, the detailed meaning thereof is described herein. Therefore, the terms used herein should be understood based on not only the name of the terms, but also the meaning of the terms and the content hereof.

In the following description, various example embodiments of the present disclosure are described in more detail with reference to the accompanying drawings. With respect to reference numerals to elements of each of the drawings, the same elements may be illustrated in other drawings, and like reference numerals may refer to like elements unless stated otherwise. The same or similar elements may be denoted by the same reference numerals even though they are depicted in different drawings. In addition, for the convenience of description, a scale and dimension of each of the elements illustrated in the accompanying drawings may be different from an actual scale and dimension, and thus, embodiments of the present disclosure are not limited to a scale and dimension illustrated in the drawings.

Before starting detailed explanations of figures, components that will be described in the specification are distinguished merely according to functions mainly performed by the components. That is, two or more components which will be described later can be integrated into a single component. Furthermore, a single component which will be explained later can be separated into two or more components. Moreover, each component which will be described can additionally perform some or all of a function executed by another component in addition to the main function thereof. Some or all of the main function of each component which will be explained can be carried out by another component. Accordingly, presence/absence of each component which will be described throughout the specification should be functionally interpreted.

The embodiment described below is a technique for selecting significant radiomic features applicable to a specific application, such as lesion classification, from medical images.

Medical images may be any one of various types of images, such as ultrasound images, X-rays, CT scans, PET scans, PET-CT scans, MRI scans, and MRA scans.

Techniques for selecting radiomic features include filter methods, wrapper methods, and embedded methods.

A filter method is a technique that evaluates the significance of features based on the statistical relationship between each feature and the subject of analysis, independent of the learning model, and selects features accordingly.

A wrapper method is a technique that repeatedly applies various combinations of features to a learning model and selects the optimal feature subset based on model performance.

An embedded method is configured so that feature selection is performed simultaneously within the training process of the learning model, and selects features based on feature importance during the model parameter optimization process.

Hereinafter, an apparatus for selecting significant radiomic features from medical images is referred to as a hardware apparatus. The hardware apparatus may be implemented in various forms. For example, the hardware apparatus may be implemented in the form of a PC, a smart device, a wearable device, a server, a cloud server, a vehicle, a robot, an IoT device, a data-processing dedicated chipset, or the like. Furthermore, it is described that the hardware apparatus may build a certain learning model using radiomic features extracted from medical images.

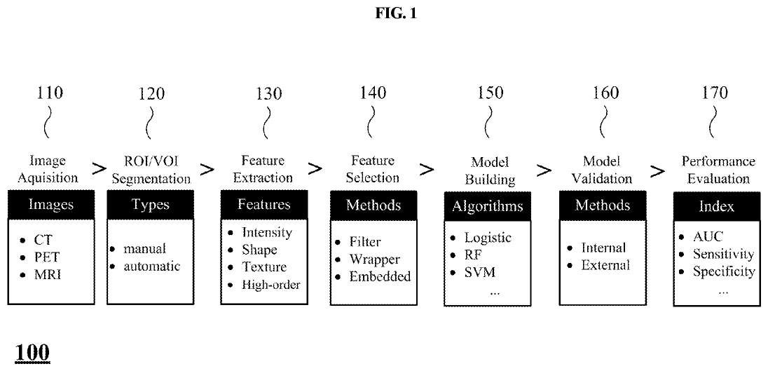

First, the process by which the hardware apparatus builds a learning model using radiomic features is described briefly. FIG. 1 is an example of a process 100 for building a learning model using radiomic features. Radiomics analysis may typically consist of seven processes.

The hardware apparatus receives medical images as input (110). The medical images may be any one of various types of images, such as CT, PET, and MRI scans.

The hardware apparatus may segment a region of interest from the medical image (120). The region of interest may be segmented manually or automatically using a deep learning-based segmentation model. Furthermore, the region of interest may be segmented using a hybrid approach that utilizes both manual and automatic techniques.

The hardware apparatus extracts radiomic features from the medical images (130). The radiomic features may be classified into categories such as shape, intensity, texture, high-order, and filter.

The hardware apparatus may select radiomic features using any one of a filter method, a wrapper method, and an embedded method (140).

The hardware apparatus may build a certain learning model using the selected radiomic features (150). The learning model may be logistic regression (LR), a decision tree, random forest (RF), XGBoost (eXtreme Gradient Boosting), LightGBM (Light Gradient Boosting Machine), CatBoost (Categorical Boosting), KNN (K-nearest neighbor), Naive Bayes, SVM (support vector machine), artificial neural network, or the like. Artificial neural networks include deep learning models such as CNN (Convolutional Neural Network).

The hardware apparatus may perform validation on the built model (160). Model validation may include internal validation and/or external validation.

Furthermore, the hardware apparatus may evaluate the performance of the model based on the validation results (170). Performance metrics may include at least one of AUC (Area Under ROC Curve), sensitivity, and specificity.

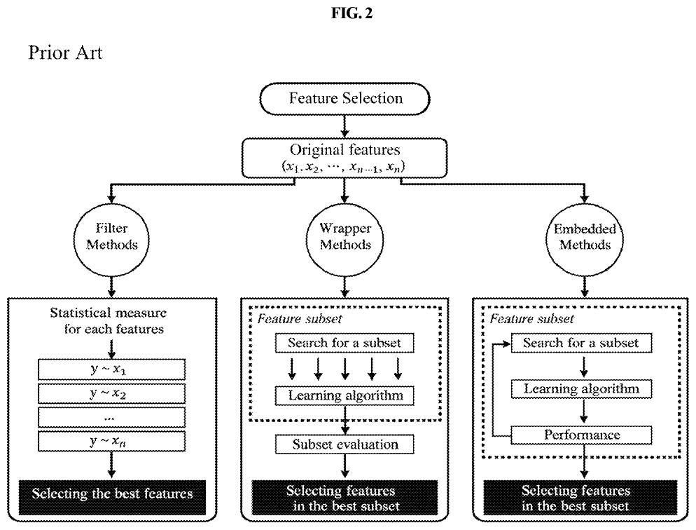

FIG. 2 is an example of a process for selecting radiomic features. The purpose of feature selection is data noise reduction, prevention of overfitting, reduction of training time and cost, and improvement of learning model performance. Radiomic feature selection techniques may be classified into filter methods, wrapper methods, and embedded methods.

A filter method screens the significance between features and an output value (outcome) to be predicted without using a separate learning algorithm. A filter method has advantages of being simple compared to other methods, being easy to use, and allowing fast calculation, but has a disadvantage of not being able to identify collinearity between features. A filter method selects features by comparing a significance indicator for a specific feature obtained through univariate analysis with a threshold value.

A wrapper method evaluates performance on all configurable feature subsets using a learning algorithm and selects the optimal feature subset. Therefore, a wrapper method takes collinearity between features into account, but has disadvantages of requiring a lot of time to find the optimal combination and being susceptible to overfitting.

An embedded method selects features using a penalty- or tree-based learning algorithm in which feature selection functionality is built into the model itself. An embedded method is more efficient than a wrapper method because it does not evaluate all subsets, but has a disadvantage of being model-dependent such that results vary depending on parameter settings.

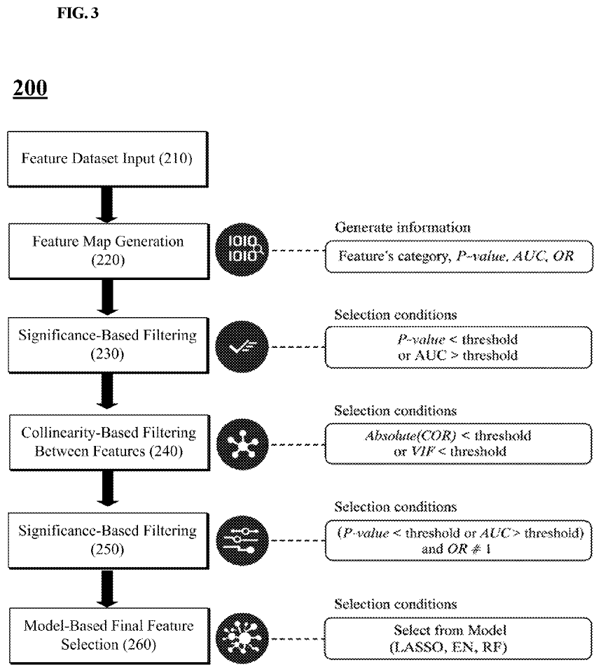

FIG. 3 is an example of a combined process 200 for selecting radiomic features.

The hardware apparatus receives radiomic features extracted from a medical image that is the subject of analysis as input (210). At this point, it is assumed that the hardware apparatus has previously secured information about the relationship between each feature and an output value to be predicted or classified by the learning model. The hardware apparatus stores the radiomic features input in an initial candidate feature set. The medical images may be any one of various types of images. The radiomic features extracted from the images may also vary depending on the type of medical image.

The hardware apparatus generates a feature map for the radiomic features included in the candidate feature set (220). The feature map stores all information computed during the feature selection process for each feature. The hardware apparatus may perform univariate analysis on the relationship between each feature and the output value. The feature map may store the category and univariate analysis results for each feature. For example, the hardware apparatus may store, in the feature map, the statistical relevance (e.g., p-value, AUC, odds ratio (OR), etc.) for each feature and basic information about the feature (category of the radiomic feature, name of the feature). Categories of radiomic features may be classified as morphological features, intensity features, texture features, and the like. Furthermore, the hardware apparatus may update the feature map during the radiomic feature selection process. Table 1 below shows examples of items stored in the feature map. The overall feature map may consist of a plurality of items from the following items for each radiomic feature.

TABLE 1

| Field Name | Description | Data Type | Example |
|---|---|---|---|
| Feature ID | Unique identifier for each radiomic feature | String/Integer | f_001 |
| Feature Name | Name of the feature | String | GLCM_entropy |
| Category | Category of the feature | String | Texture |
| p-value | Statistical significance with respect to the output value | Float | 0.012 |
| AUC | Predictive performance indicator of the feature | Float | 0.78 |
| Odds Ratio (OR) | Degree of influence on the output value | Float | 1.45 |
| COR | Maximum correlation coefficient with other features | Float | 0.85 |
| VIF | Multicollinearity indicator | Float | 5.2 |
| Selected Step1 | Whether the first filtering is passed | Boolean | True |
| Selected Step2 | Whether the second filtering is passed | Boolean | False |
| Selected Step3 | Whether the third filtering is passed | Boolean | True |
| Representative Flag | Whether the feature is representative within a collinear group | Boolean | True |
| Final Selected | Whether the feature is finally selected | Boolean | True |

The feature map may store, for each radiomic feature, the basic radiomic information, statistical indicators with respect to the output value (p-value, AUC, OR), collinearity indicators, and whether the feature passed (was selected at) each filtering stage. The hardware apparatus reuses previously computed information in the feature map and to avoid redundant computations during the filtering process. This allows the hardware apparatus to efficiently perform combined filtering on high-dimensional radiomic feature sets (hundreds to thousands of features).

The hardware apparatus performs filtering on the radiomic features included in the candidate feature set based on the significance of each feature (230). The hardware apparatus examines the significance of each radiomic feature included in the candidate feature set based on the relationship between the feature and the output value. The hardware apparatus may filter (remove) features with low relevance to the output value. The hardware apparatus may determine the significance of each feature based on the p-value or AUC and may remove features with low significance from the candidate feature set. For example, the hardware apparatus may remove from the candidate feature set features for which the p-value is equal to or greater than a first threshold (e.g., 0.5) or the AUC is less than a second threshold (e.g., 0.7). That is, the hardware apparatus may retain in the candidate feature set only features with high correlation with the output value of the model based on the p-value or AUC.

The hardware apparatus performs filtering on the radiomic features remaining in the candidate feature set based on collinearity between features (240). Accordingly, the hardware apparatus may compute indicators representing the degree of correlation between features. For example, the hardware apparatus may compute the absolute correlation (e.g., Correlation Coefficient such as Pearson correlation) considering only the relationships between variables. In addition, the hardware apparatus may compute the VIF (Variance Inflation Factor) considering only the relationships among independent variables. The hardware apparatus may remove from the candidate feature set features for which the absolute correlation is equal to or greater than a third threshold or the VIF is equal to or greater than a fourth threshold. That is, the hardware apparatus may retain in the candidate feature set only independent features based on the absolute correlation or VIF.

The hardware apparatus may identify groups of radiomic features whose collinearity is equal to or greater than a threshold. The hardware apparatus may select a representative feature from a group of features with high collinearity according to predefined criteria and remove the remaining features. The representative feature may be selected based on statistical significance, predictive performance, effect size, or a combination thereof.

The hardware apparatus may perform second filtering on the radiomic features remaining after the first removal to eliminate collinearity between features. In one embodiment, the hardware apparatus may compute an absolute correlation (COR) between features within each feature category, and generate a collinearity group consisting of features whose absolute correlation is equal to or greater than a preset threshold. The hardware apparatus may select a representative feature from within the collinearity group based on statistical relevance or predictive performance, and remove the remaining features. In addition, after the absolute correlation-based filtering, the hardware apparatus may compute a VIF (Variance Inflation Factor) for each feature, and further eliminate multicollinearity among multiple features by removing features whose VIF is equal to or greater than a threshold.

The hardware apparatus may classify radiomic features into categories and filter features within each category based on absolute correlation or VIF. Radiomic feature categories may be classified into morphological features (volume, sphericity, etc.), intensity features (mean brightness, skewness, entropy, etc.), texture features, filter features, and the like.

The hardware apparatus may perform significance-based filtering on each of the radiomic features remaining in the candidate feature set based on the relationship between the feature and the output value (250). At this point, the hardware apparatus may select features based on the odds ratio together with “p-value or AUC.” For example, the hardware apparatus may remove from the candidate feature set features for which (i) the p-value is equal to or greater than a fifth threshold or the AUC is less than a sixth threshold, and (ii) the odds ratio (OR) is 1 (i.e., features that do not affect the output regardless of variation). In one embodiment, the hardware apparatus may remove features having an odds ratio close to 1 (e.g., an odds ratio within a predefined numerical range including 1). That is, the hardware apparatus may retain in the candidate feature set only features that have high correlation with the output value of the learning model based on “p-value or AUC” and whose variable value changes affect the learning model.

In the process 250, the hardware apparatus may select features based on the significance of each radiomic feature. In one embodiment, the significance comprises at least one of p-value, AUC and an odds ratio.

Finally, the hardware apparatus may apply an embedded method to the radiomic features remaining in the candidate feature set to select the final radiomic features (260). For example, the hardware apparatus may select the final radiomic features using a learning model such as LASSO, EN (Elastic-Net), or RF. The hardware apparatus may select a certain number of features ranked at the top in feature importance evaluation using RF as the final radiomic features.

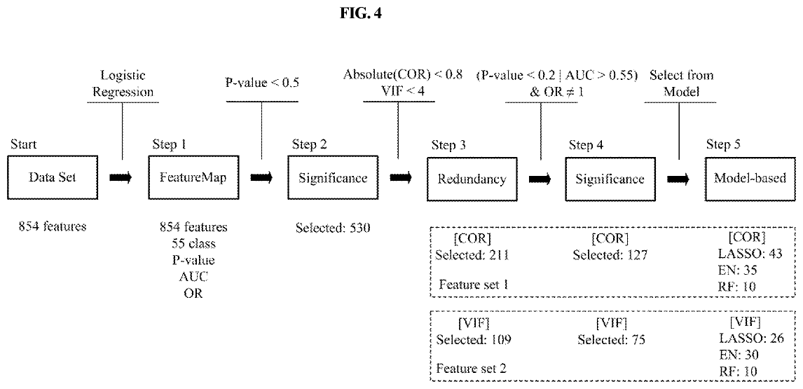

The performance of the aforementioned radiomic feature selection technique is validated. FIG. 4 illustrates a radiomic feature selection process using an actual dataset.

Dataset 1 includes radiomic features extracted from PET-CT images of patients with metabolic syndrome collected at Soonchunhyang University Seoul Hospital. Dataset 1 is used for building a model for predicting metabolic syndrome after bariatric surgery. The sample size of Dataset 1 is 79 subjects, and it consists of 854 features across 55 categories in total.

A training dataset is selected from 70% of the entire dataset for model building and the remaining 30% is a validation dataset. The learning models include LR, RF, and SVM. And model performance was evaluated using accuracy, sensitivity, specificity, BA (balanced accuracy), and AUC. BA was used to determine the optimal cutoff of the predictive model.

The hardware apparatus generated a feature map having a p-value, AUC, and OR for each of the 854 features. The statistical values for the features may be information included in the dataset in advance, or values computed by the hardware apparatus through univariate analysis of the features.

The process of selection of significant radiomic features described in FIG. 3. In this process, the first threshold for the p-value is set as 0.5 in process 230, the third threshold for the absolute Correlation Coefficient (COR) is set as 0.8 and the third threshold for VIF is set as 4 in process 240, the fifth threshold for the p-value is set as 0.2 and the sixth threshold for AUC is set as 0.55 in process 250, and the top-ranking criterion is set as 10 in process 260.

(1) The hardware apparatus retained 530 features with a p-value<0.5 from among the 854 features in the feature set. (2) The hardware apparatus then removed redundant features based on absolute COR and VIF. The hardware apparatus retained 211 features in Feature Set 1 based on the criterion of absolute COR<0.8. The hardware apparatus also retained 109 features in Feature Set 2 based on the criterion of VIF<4. (3) The hardware apparatus then selected feature values for which (i) p-value<0.2 or AUC>0.55, and (ii) OR≠1. In this process, Feature Set 1 had 127 features remaining, and Feature Set 2 had 75 features remaining. (iv) Finally, the hardware apparatus selected the final radiomic features using an embedded method with LASSO, EN, and RF, respectively. From Feature Set 1, LASSO selected 43 features, EN selected 35 features, and RF selected 10 features. From Feature Set 2, LASSO selected 26 features, EN selected 30 features, and RF selected 10 features. The top-ranking criterion for RF was set to 10. Table 2 below shows the performance of models LR, RF, and SVM built by cross-validation using the feature sets.

TABLE 2

| Model | | | No. of | Performance Indicator | | | | |
|---|---|---|---|---|---|---|---|---|
| Type | Feature Set | | Features | AUC | Accuracy | Sensitivity | Specificity | BA |
| LR | COR | LASSO | 43 | 0.479 | 0.522 | 0.625 | 0.467 | 0.546 |
| | (Feature Set 1) | EN | 35 | 0.558 | 0.652 | 0.500 | 0.733 | 0.617 |
| | | RF | 10 | 0.800 | 0.826 | 0.750 | 0.867 | 0.808 |
| | VIF | LASSO | 26 | 0.508 | 0.565 | 0.625 | 0.533 | 0.579 |
| | (Feature Set 2) | EN | 30 | 0.442 | 0.652 | 0.000 | 1.000 | 0.500 |
| | | RF | 10 | 0.792 | 0.870 | 0.625 | 1.000 | 0.812 |
| RF | COR | LASSO | 43 | 0.587 | 0.739 | 0.500 | 0.867 | 0.683 |
| | (Feature Set 1) | EN | 35 | 0.546 | 0.739 | 0.250 | 1.000 | 0.625 |
| | | RF | 10 | 0.675 | 0.652 | 0.750 | 0.600 | 0.675 |
| | VIF | LASSO | 26 | 0.667 | 0.826 | 0.500 | 1.000 | 0.750 |
| | (Feature Set 2) | EN | 30 | 0.650 | 0.783 | 0.500 | 0.933 | 0.717 |
| | | RF | 10 | 0.662 | 0.826 | 0.500 | 1.000 | 0.750 |
| SVM | COR | LASSO | 43 | 0.650 | 0.826 | 0.625 | 0.933 | 0.779 |
| | (Feature Set 1) | EN | 35 | 0.650 | 0.739 | 0.625 | 0.800 | 0.713 |
| | | RF | 10 | 0.692 | 0.652 | 0.875 | 0.533 | 0.704 |
| | VIF | LASSO | 26 | 0.692 | 0.739 | 0.625 | 0.800 | 0.713 |
| | (Feature Set 2) | EN | 30 | 0.717 | 0.783 | 0.500 | 0.933 | 0.717 |
| | | RF | 10 | 0.783 | 0.826 | 0.625 | 0.933 | 0.779 |

Referring to Table 2, the LR model showed the highest performance with an AUC of 0.8 for radiomic features selected using COR and RF, the RF model showed the highest performance with an AUC of 0.675 for radiomic features selected using COR and RF, and the SVM model showed the highest performance with an AUC of 0.783 for radiomic features selected using VIF and RF.

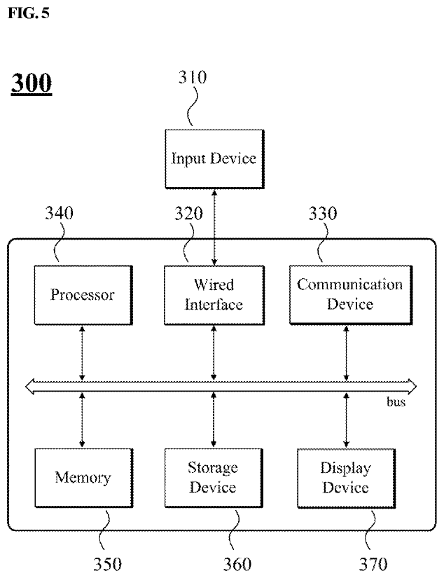

FIG. 5 is an example of a hardware apparatus 300 for selecting radiomic features. The hardware apparatus 300 may be physically implemented in various forms. For example, the hardware apparatus 300 may have forms such as a computer device such as a PC, a server of a network, and a dedicated chipset for data processing.

The hardware apparatus 300 may include an input device 310, a wired interface 320, a communication device 330, a processor 340, a memory 350, and a storage device 360. Alternatively, the hardware apparatus 300 may include the input device 310, the wired interface 320, the communication device 330, the processor 340, the memory 350, the storage device 360, and a display device 370.

The internal components of the hardware apparatus 300 may be connected by a bus. A specific bus may be used depending on the type of entity being connected. For example, the bus may be any one of AMBA (AHB/AXI/APB), PCIe, SPI (Serial Peripheral Interface), or MIPI (Mobile Industry Processor Interface).

The input device 310 is a device that receives user commands or information. The input device 310 may receive a specific medical image. The medical images may be any one of various types of images, such as ultrasound images, X-rays, CT scans, PET scans, PET-CT scans, MRI scans, and MRA scans. The input device 310 may also receive all radiomic features extracted from a specific medical image. The input device 310 may be at least one of a mouse, a keyboard, a touch input device, a camera, a SCSI (Small Computer System Interface) device, a PCI (Peripheral Component Interconnect) bus-based device, or an ATAPI (ATA Packet Interface) device.

The wired interface 320 is a device component that delivers data transmitted by the input device 310 to the inside of the apparatus. The wired interface 320 may be composed of software drivers and hardware. The wired interface 320 may include a controller corresponding to each input device, a device driver that controls the operation of the controller, and a kernel I/O subsystem that comprehensively manages input/output control requests of the device driver. The kernel I/O subsystem stores input/output requests from the device driver in a queue and schedules the requests based on request priority or device status. The wired interface 320 may include interfaces such as PS/2, USB (Universal Serial Bus), an Ethernet port, HDMI, MIPI CSI, DisplayPort, and Thunderbolt.

The communication device 330 refers to a component that receives and transmits certain information through an external wired or wireless network. The communication device 330 may be composed of a circuit including an antenna and a communication module (S/W module, chip, etc.) corresponding to a communication protocol. The communication protocol may be at least one of wired LAN (Ethernet), wireless LAN (IEEE 802.11), mobile communication (LTE, 5GNR, etc.), Bluetooth, and NFC.

The processor 340 controls operations of overall components of the hardware apparatus 300. The processor 340 is a general-purpose processor that executes at least a portion of a control program installed in the storage device 360 or at least a portion of a program loaded in the memory 350. The processor 340 may be implemented as circuitry such as a system on chip (SoC) or an integrated circuit (IC). The processor 340 may include a combination of one or more processors such as a CPU (central processing unit), an MPU (microprocessor unit), an MCU (micro controller unit), a GPU (graphic processing unit), an NPU (neural processing unit), a DSP (digital signal processor), an AP (application processor), or a CP (communication processor).

The memory 350 may store data generated during the radiomic feature filtering process. For example, the memory 350 may store a feature map that is updated during the radiomic feature filtering process. The memory 350 is a volatile memory such as a DRAM or an SRAM.

The storage device 360 may store a program for performing radiomic feature filtering, a specific medical image, all radiomic features extracted from a specific medical image, a feature map that is updated during the radiomic feature filtering process, and the finally selected radiomic features. The storage device 360 may be implemented as a device such as a hard disk drive, a solid state drive, a USB flash drive, a memory card, an optical disk, or a network-based storage device (Network Attached Storage, cloud storage, etc.).

The display device 370 may output an interface screen for radiomic feature filtering, initial radiomic features, finally selected radiomic features, and the like. The display device 370 may be implemented in various display methods such as liquid crystal, plasma, light-emitting diode, organic light-emitting diode, surface-conduction electron-emitter, carbon nano-tube, and nano-crystal.

The processor 340 may execute a program stored in the storage device 360 to perform radiomic feature filtering. The processor 340 may extract radiomic features from medical images input through the input device 310 or the wired interface 320. The extracted radiomic features may be classified into categories such as shape, intensity, texture, high-order, and filter.

The processor 340 performs univariate analysis on each of the extracted radiomic features with respect to the output value of the learning model to compute statistical indicators. The statistical indicators may include a p-value, AUC, and odds ratio (OR).

The processor 340 may generate a feature map including the computed statistical relevance indicators. As shown in Table 1, the feature map may store the radiomic feature identifier, statistical relevance indicators, whether a candidate feature is selected, and the like. Whether a candidate feature is selected indicates whether the feature has been selected as a significant feature at the corresponding time step during the filtering process. The processor 340 updates whether a candidate feature is selected while proceeding with the filtering process. The candidate feature set consists of the radiomic features selected as candidate features in the feature map. Accordingly, the finally selected radiomic features consist of the radiomic features remaining as candidate features in the feature map after the filtering process is completed.

The processor 340 may perform first filtering on each of the radiomic features included in the initial candidate feature set based on the relevance to the output value. Specifically, the processor 340 may remove from the candidate feature set features for which the p-value is equal to or greater than a first threshold (e.g., 0.5) or the AUC is less than a second threshold (e.g., 0.7). A lenient threshold may be used in the first filtering. The processor 340 primarily removes features that have low correlation with the output value and are unlikely to contribute to building the learning model.

The processor 340 may perform second filtering on the radiomic features remaining in the candidate feature set after the first filtering based on collinearity between features. The processor 340 may compute the absolute Correlation Coefficient (COR) and/or the Variance Inflation Factor (VIF). The processor 340 may remove from the candidate feature set features for which the absolute correlation is equal to or greater than a third threshold (e.g., 0.8) or the VIF is equal to or greater than a fourth threshold (e.g., 4). At this point, the processor 340 identifies a “redundant feature group” consisting of radiomic features whose collinearity is equal to or greater than a threshold, selects one representative feature from the redundant feature group according to predefined criteria such as statistical significance, predictive performance, or effect size, and removes the remaining features. Meanwhile, the processor 340 may classify radiomic features by category and perform collinearity-based filtering within each category.

The processor 340 may perform the second filtering on the radiomic features as follows. In one embodiment, the processor 340 may compute an absolute correlation (COR) between features within each feature category, and generate a collinearity group consisting of features whose absolute correlation is equal to or greater than a preset threshold. The processor 340 may select a representative feature from within the collinearity group based on statistical relevance or predictive performance, and remove the remaining features. In addition, after the absolute correlation-based filtering, the processor 340 may compute a VIF (Variance Inflation Factor) for each feature, and further eliminate multicollinearity among multiple features by removing features whose VIF is equal to or greater than a threshold.

The processor 340 may perform third filtering on the radiomic features remaining in the candidate feature set after the second filtering based on the relevance to the output value and the odds ratio (OR). For example, the processor 340 may remove from the candidate feature set features for which (i) the p-value is equal to or greater than a fifth threshold (e.g., 0.2) or the AUC is less than a sixth threshold (e.g., 0.55), and (ii) the odds ratio is 1. In one embodiment, the processor 340 may remove features having an odds ratio of approximately 1 (e.g., an odds ratio within a predefined numerical range including 1).

The processor 340 may apply an embedded method to the radiomic features remaining in the candidate feature set after the third filtering to select the final radiomic features. Learning models such as LASSO, EN, and RF may be used as the embedded method. For example, the processor 340 may select a certain number of features (e.g., the top 10 features) ranked at the top in feature importance evaluation using RF as the final radiomic features.

The processor 340 may build a learning model such as logistic regression (LR), random forest (RF), and support vector machine (SVM) using the selected final radiomic features. The processor 340 may load instructions for performing radiomic feature filtering from the storage device 360 into the memory 350.

The processor 340 may execute the instructions to perform the following operations: (i) extracting a plurality of radiomic features from the medical images; (ii) performing univariate statistical analysis on each of the radiomic features with respect to an output value of a learning model to compute the statistical relevance indicators; (iii) performing a first removal of features whose relevance to the output value is below a threshold, from a candidate radiomic feature set including the radiomic features, based on at least one of the statistical relevance indicators; (iv) performing a second removal of redundant features from the candidate radiomic feature set remaining after the first removal based on collinearity criteria; (v) performing a third removal of features whose relevance to the output value is below a threshold, from the candidate radiomic feature set remaining after the second removal, based on at least one of the statistical relevance indicators; and (vi) applying an embedded method to the candidate radiomic feature set remaining after the third removal to select the final radiomic features.

Hereinafter, an electronic device is described as a device that performs inference using a learning model. The electronic device may have forms such as a computer device, a server, a smart device, a vehicle, a robot, and a data-processing dedicated chipset. The electronic device performs inference using a specific learning model. The specific learning model is a model trained in advance using radiomic features extracted through the processes described in FIGS. 3 through 5.

The electronic device may receive a medical image of a patient from a medical imaging device or an EMR (Electronic Medical Record). The electronic device may extract radiomic features from the medical image. The electronic device may use all radiomic features or selected significant radiomic features as input data for the learning model. The electronic device computes certain clinical information based on the inference result of the learning model. The clinical information may be one of various types of information, such as the health status of the patient, whether the patient has a specific disease, whether a tumor has metastasized, and responsiveness to anticancer treatment.

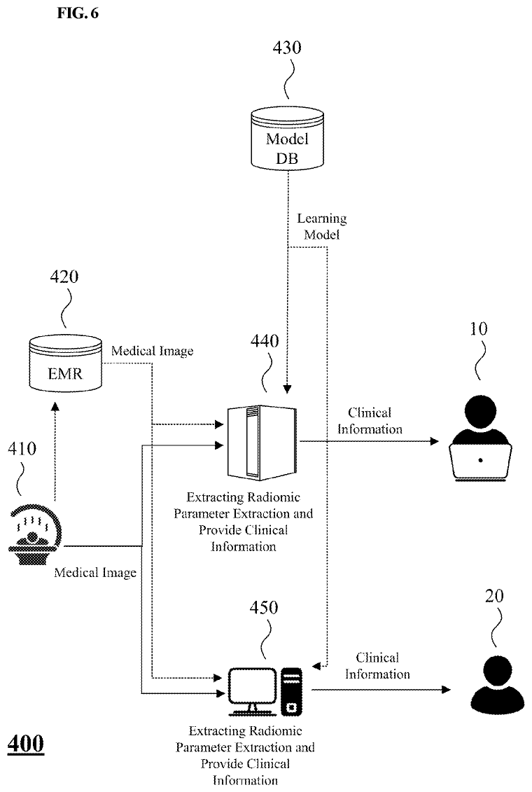

FIG. 6 is an example of a subject diagnosis system 400 using a learning model that takes radiomic features as input data. In FIG. 6, the electronic device takes the form of a server 440 and a computer terminal 450. The diagnosis system 400 may include a medical imaging device 410, an EMR 420, a model database (DB) 430, and a server 440. Alternatively, the diagnosis system 400 may include the medical imaging device 410, the EMR 420, the model DB 430, and a computer terminal 450.

The medical imaging device 410 generates a medical image for a specific patient and may be one of an X-ray machine, a CT scanner, an MRI scanner, and a PET scanner. The EMR 420 may receive and store a medical image from the medical imaging device 410 and may provide the stored medical image upon request from the server 440 or the computer terminal 450.

The model DB 430 stores a learning model trained with significant radiomic features extracted by the radiomic feature filtering method described above. The type of learning model may be various, such as logistic regression (LR), random forest (RF), SVM, and XGBoost. The diseases predicted or classified by the learning model may include cancer, metabolic syndrome, cardiovascular disease, and neurological disease. The model DB 430 may also store information on the significant radiomic features selected during the radiomic feature filtering process.

The server 440 may receive a specific learning model from the model DB 430 and receive a medical image of a patient from the medical imaging device 410 or the EMR 420. The server 440 may extract radiomic features from the medical image, use the significant radiomic features as input data for the learning model, generate clinical information based on the inference result, and provide it to a user 10.

The computer terminal 450 may receive a specific learning model from the model DB 430 and receive a medical image of a patient from the medical imaging device 410 or the EMR 420. The computer terminal 450 may extract radiomic features from the medical image, use the significant radiomic features as input data for the learning model, generate clinical information based on the inference result, and provide it to a user 20.

Users 10 and 20 may utilize the prediction results and clinical information to perform various clinical decisions, such as diagnosing patient conditions, establishing treatment plans, and predicting prognoses. The users 10 and 20 may perform the following treatment actions or establish treatment plans based on the clinical information, and the electronic device may also generate treatment choices or plans by utilizing AI such as an LLM (Large Language Model).
(1) Selection of Treatment Modality: The users 10 and 20 may select a treatment modality including at least one of surgical treatment, drug treatment, or radiation therapy based on a severity classification result included in the clinical information.
(2) Selection of Treatment Drug and Determination of Dosage: The users 10 and 20 may determine the type and dosage of a drug to be administered based on a treatment response prediction result included in the clinical information.
(3) Establishment of Post-Treatment Monitoring Plan: The users 10 and 20 may establish a monitoring plan including follow-up intervals and additional examination items based on a prognosis prediction result included in the clinical information.

Furthermore, the radiomic feature selection technique, the method for building a learning model using the selected radiomic features, and the method for computing clinical information using the learning model may be implemented as a program (or application) including an executable algorithm that may be executed on a computer. The program may be stored in and provided on a temporary or non-transitory computer readable medium.

The non-transitory computer readable medium refers to a medium that stores data semi-permanently and is capable of being read by a device, rather than a medium that stores data for a short period of time, such as a register, cache, or memory. Specifically, the various applications or programs described above may be provided by being stored in the non-transitory computer readable medium such as a CD, a DVD, a hard disk, a Blu-ray disk, a USB, a memory card, a ROM, a PROM, an EPROM, an EEPROM, or a flash memory.

The transitory computer readable medium refers to various types of RAM such as a static RAM (SRAM), a dynamic RAM (DRAM), a synchronous DRAM (SDRAM), a double data rate SDRAM (DDR SDRAM), an enhanced SDRAM (ESDRAM), a synclink DRAM (SLDRAM), and a direct Rambus RAM (DRRAM).

The present embodiments and the drawings appended hereto merely show some of the technical ideas included in the technology described above clearly, and it is obvious that all modifications and specific embodiments that may be easily inferred by those skilled in the art within the scope of the technical ideas are included in the scope of rights of the technology described above.

What is claimed is:

1. A method for supporting patient treatment using a learning model, the method comprising:
receiving, by an electronic device, a medical image of a patient;
extracting, by the electronic device, radiomic features from the medical image;
inputting, by the electronic device, the extracted radiomic features into a pre-trained learning model to generate clinical information about the patient;
providing, by the electronic device, the clinical information to a user terminal; and
supporting selection of a treatment modality for the patient or establishing a treatment plan based at least in part on the clinical information,
wherein the learning model is trained using significant radiomic features, and
wherein the significant radiomic features are selected through a process comprising:
(i) a first filtering based on a filter method applied to radiomic features extracted from medical images as training data;
(ii) a second filtering that removes redundant features based on collinearity from candidate radiomic features remaining after the first filtering;
(iii) a third filtering based on a filter method applied to candidate radiomic features remaining after the second filtering; and
(iv) selecting radiomic features based on an embedded method applied to candidate radiomic features remaining after the third filtering.

2. The method of claim 1, wherein the electronic device selects, from all radiomic features extracted from the medical image, only the radiomic features corresponding to the significant radiomic features used during the training process of the learning model, and inputs the selected radiomic features into the learning model.

3. A method for building a learning model using radiomic features, the method comprising:
receiving, by a hardware apparatus, a plurality of radiomic features extracted from medical images;
generating, by the hardware apparatus, a feature map including one or more statistical relevance indicators and one or more collinearity indicators for each of the radiomic features;
selecting, by the hardware apparatus, significant radiomic features by removing some of the radiomic features using the feature map; and
training, by the hardware apparatus, a learning model using the significant radiomic features,
wherein selecting the significant radiomic features comprises:
performing, by the hardware apparatus, univariate statistical analysis on each of the radiomic features with respect to an output value of the learning model to compute the one or more statistical relevance indicators;
performing, by the hardware apparatus, a first removal of features whose relevance to the output value is below a threshold, from a candidate radiomic feature set including the radiomic features, based on at least one first statistical relevance indicator selected from the one or more statistical relevance indicators;
performing, by the hardware apparatus, a second removal of redundant features from the candidate radiomic feature set remaining after the first removal based on the one or more collinearity indicators;
performing, by the hardware apparatus, a third removal of features whose relevance to the output value is below a threshold, from the candidate radiomic feature set remaining after the second removal, based on at least one second statistical relevance indicator selected from the one or more statistical relevance indicators; and
applying, by the hardware apparatus, an embedded method to the candidate radiomic feature set remaining after the third removal to select final radiomic features.

4. The method of claim 3, wherein the one or more statistical relevance indicators comprise at least one of a p-value and AUC (Area Under ROC Curve).

5. The method of claim 3, wherein performing the second removal comprises:
identifying, by the hardware apparatus, a redundant feature group consisting of radiomic features, from the candidate radiomic feature set remaining after the first removal, for which an absolute correlation is equal to or greater than a first threshold or a VIF (Variance Inflation Factor) is equal to or greater than a second threshold; and
removing, by the hardware apparatus, radiomic features other than a representative radiomic feature from the redundant feature group.

6. The method of claim 5, wherein the representative radiomic feature is the radiomic feature having the best statistical relevance indicator value or the best predictive performance indicator value among the redundant feature group.

7. The method of claim 3, wherein performing the third removal comprises:
removing, by the hardware apparatus, from the candidate radiomic feature set remaining after the second removal, a radiomic feature for which (i) a p-value is equal to or greater than a third threshold or an AUC (Area Under ROC Curve) is less than a fourth threshold, and (ii) an odds ratio is 1 or an odds ratio within a predefined numerical range including 1.

8. The method of claim 3, wherein the threshold used in the first removal is set to a value that maintains more radiomic features than the threshold used in the third removal.

9. A hardware apparatus comprising:
a storage device configured to store medical images;
a memory configured to store instructions for radiomic feature filtering; and
a processor configured to execute the instructions to:
(i) extract a plurality of radiomic features from the medical images;
(ii) perform univariate statistical analysis on each of the radiomic features with respect to an output value of a learning model to compute a plurality of statistical relevance indicators;
(iii) perform a first removal of features whose relevance to the output value is below a threshold, from a candidate radiomic feature set including the radiomic features, based on at least one first statistical relevance indicator selected from the plurality of statistical relevance indicators;
(iv) perform a second removal of redundant features from the candidate radiomic feature set remaining after the first removal based on collinearity criteria;
(v) perform a third removal of features whose relevance to the output value is below a threshold, from the candidate radiomic feature set remaining after the second removal, based on at least one second statistical relevance indicator selected from the plurality of statistical relevance indicators, wherein the at least one second statistical relevance indicator comprises the at least one first statistical relevance indicator; and
(vi) apply an embedded method to the candidate radiomic feature set remaining after the third removal to select final radiomic features.

10. The hardware apparatus of claim 9, wherein the plurality of statistical relevance indicators comprise a p-value, AUC (Area Under ROC Curve) and an odds ratio.

11. The hardware apparatus of claim 9, wherein, in performing the second removal, the processor is configured to:
identify a redundant feature group consisting of radiomic features, from the candidate radiomic feature set remaining after the first removal, for which an absolute correlation is equal to or greater than a first threshold or a VIF (Variance Inflation Factor) is equal to or greater than a second threshold; and
remove radiomic features other than a representative radiomic feature from the redundant feature group.

12. The hardware apparatus of claim 11, wherein the representative radiomic feature is the radiomic feature having the best statistical relevance indicator value or the best predictive performance indicator value among the redundant feature group.

13. The hardware apparatus of claim 9, wherein, in performing the third removal, the processor is configured to remove, from the candidate radiomic feature set remaining after the second removal, a radiomic feature for which (i) a p-value is equal to or greater than a third threshold or an AUC (Area Under ROC Curve) is less than a fourth threshold, and (ii) an odds ratio is 1 or an odds ratio within a predefined numerical range including 1.

14. The hardware apparatus of claim 9, wherein the threshold used in the first removal is set to a value that maintains more radiomic features than the threshold used in the third removal.
